(19) United States
(12) Patent Application Publication
(10) Pub. No.: US 2025/0174339 A1
(43) Pub. Date: May 29, 2025

(54) ENHANCED PARALLEL OPERATION OF USER INTERFACE FOR CARDIAC INDEX DETERMINATION

(71) Applicant: Cathworks Ltd., Kfar Saba (IL)

(72) Inventors: Ramin Mousavi, Kfar Saba (IL); Vered Anin, Kfar Saba (IL); Hila Meirovitch, Kfar Saba (IL)

(21) Appl. No.: 18/957,515

(22) Filed: Nov. 22, 2024

Related U.S. Application Data
(60) Provisional application No. 63/603,038, filed on Nov. 27, 2023.
(60) Provisional application No. 63/603,570, filed on Nov. 28, 2023.

(51) Int. Cl. G16H 30/40; G06T 7/00

(52) U.S. Cl. CPC G16H 30/40; G06T 7/0012; G06T 2200/24; G06T 2207/30096; G06T 2207/30104

(57) ABSTRACT

Systems and methods for enhanced parallel operation of user interface for cardiac index determination. An example method includes accessing medical images depicting a portion of a patient's heart. A unified user interface is presented which allows for a user to select medical images for analysis, with the unified user interface allowing a user to select a lesion and adjust detected vessels on a first medical image while the system is detecting vessels on a different medical image displayed on the unified user interface without navigating to a later user interface.

### CROSS-REFERENCE TO RELATED APPLICATIONS

This application claims priority to U.S. Prov. Patent App. No. 63/603,038 titled “ENHANCED PARALLEL OPERATION OF USER INTERFACE FOR CARDIAC INDEX DETERMINATION” and filed on Nov. 27, 2023, and U.S. Prov. Patent App. No. 63/603,570 titled “ENHANCED PARALLEL OPERATION OF USER INTERFACE FOR CARDIAC INDEX DETERMINATION” and filed on Nov. 28, 2023, the disclosures of which are hereby incorporated herein by reference in their entireties.

### BACKGROUND

Cardiovascular disease (CVD) is a leading cause of morbidity and mortality, with an estimated 244.1 million people worldwide with CVD, particularly due to the subsect of CVD, coronary artery disease (CAD). CAD may involve a prolonged asymptomatic developmental phase, with clinical manifestations often resulting in angina pectoris, acute myocardial infarction (MI), or cardiac death. The underlying mechanism that may cause CAD involves atherosclerosis of the coronary arteries. Atherosclerosis is a plaque buildup that narrows the coronary arteries and decreases blood flow to the heart, resulting in ischemia or coronary stenosis. Revascularization is the preferred therapy for patients with moderate to severe ischemia or stenosis, resulting in significant improvements for the patient. Revascularization strategies include many techniques such as open-heart surgery, coronary artery bypass grafting (CABG), and percutaneous coronary intervention (PCI) methods such as balloon angioplasty, bare-meta stents (BMS), and first- and second-generation drug-eluting stents (DES). The severity of CAD can be assessed through vascular computer models.

### BRIEF DESCRIPTION OF THE DRAWINGS

FIG. 1A is a block diagram of an example angiogram analysis system generating a user interface.

FIG. 1B is a block diagram of the example angiogram analysis system updating the user interface based on user input.

FIGS. 2A-2I illustrate example user interfaces associated with parallel adjusting of angiograms.

FIG. 3 is a flowchart of an example process for parallel adjusting of angiograms prior to generation of an index indicative of vascular function and/or three-dimensional cardiac model.

### DETAILED DESCRIPTION

### Overview

This specification describes techniques to increase an ease, and reliability, at which a medical professional can generate an index indicative of vascular function and/or three-dimensional cardiac model associated with a patient. As will be described, a threshold number of cardiac images (e.g., angiographic images, also referred to herein as angiograms) may be obtained which depict a portion of a patient's heart. These cardiac images may depict the portion from at different viewpoints, such that a system may generate a three-dimensional model of the portion. The system may additionally determine the above-described index, which in some embodiments may be a fractional flow reserve (FFR) value. A medical professional may leverage user interfaces described herein to identify one or more lesions which are to be analyzed. As will be described, in contrast to prior systems and techniques, the medical professional may leverage simplified user interfaces which increase the parallel operation of tasks, and user input, required to cause the system to generate the index and/or three-dimensional model.

The medical professional may access the threshold number of medical images via a user interface. For example, in some embodiments the system described herein may access three medical images. The threshold number of images may, in some embodiments, be automatically selected from a set of images. As an example, at each viewpoint there may be a substantial number of medical images. For this example, the viewpoint may represent an orientation of an imaging system (e.g., a c-arm) and the imaging system may obtain multiple images while at the orientation. The system described herein may, in some embodiments, automatically select a particular medical image at each viewpoint. For example, different criteria may be used to select the particular medical image (e.g., an optimal image). Further description related to automatic selection is included in U.S. Patent Pub. 2023/0252632, which is hereby incorporated herein by reference in its entirety.

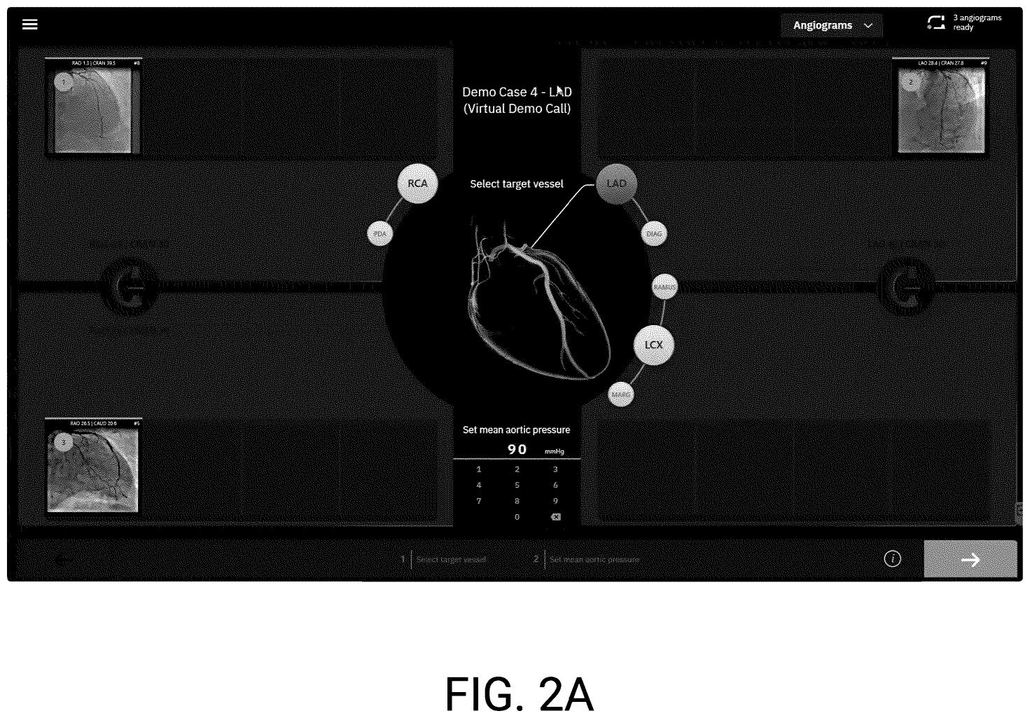

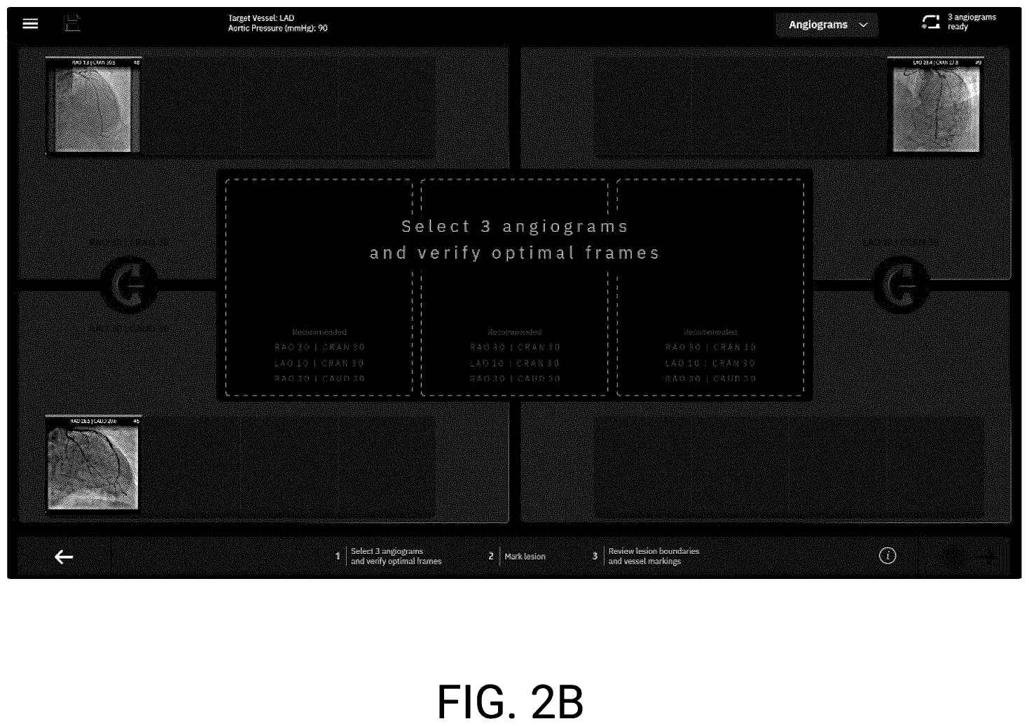

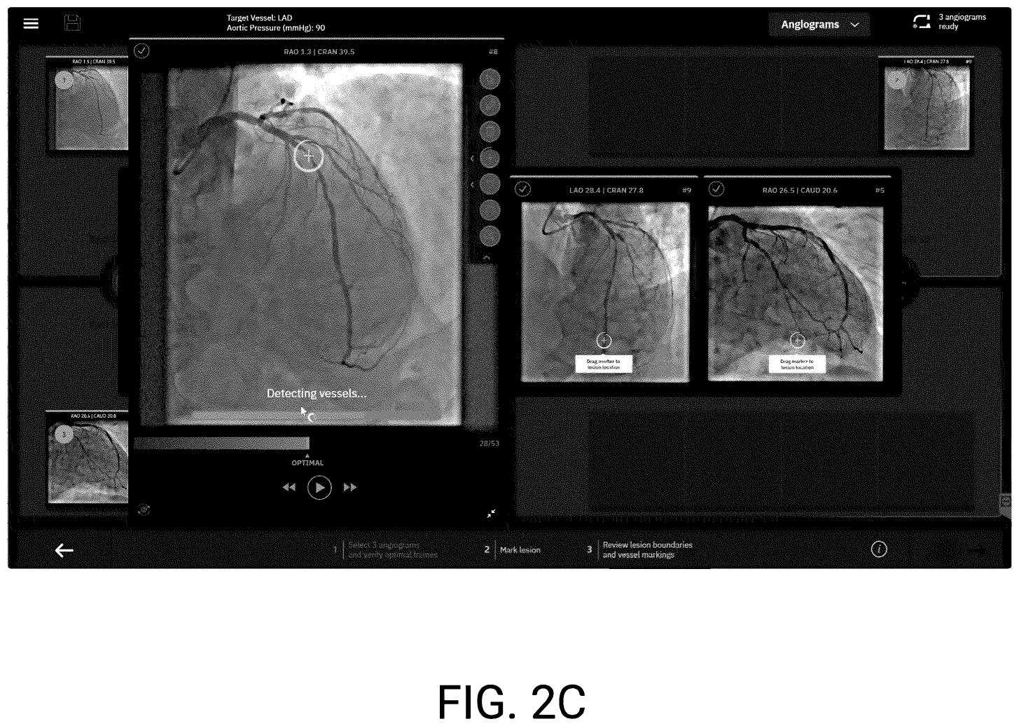

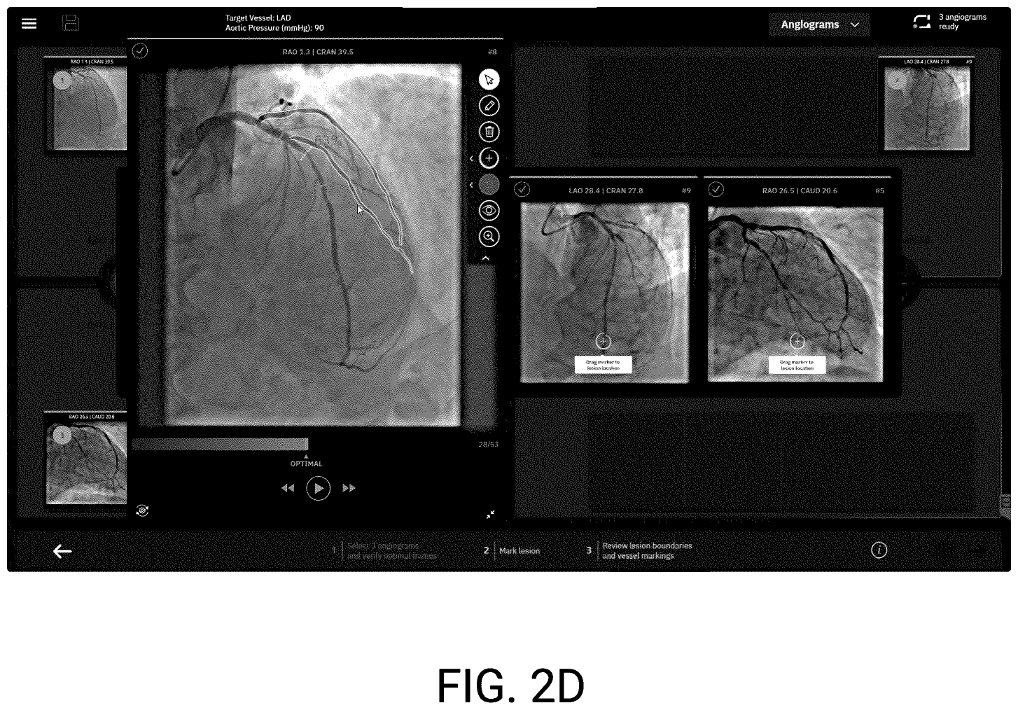

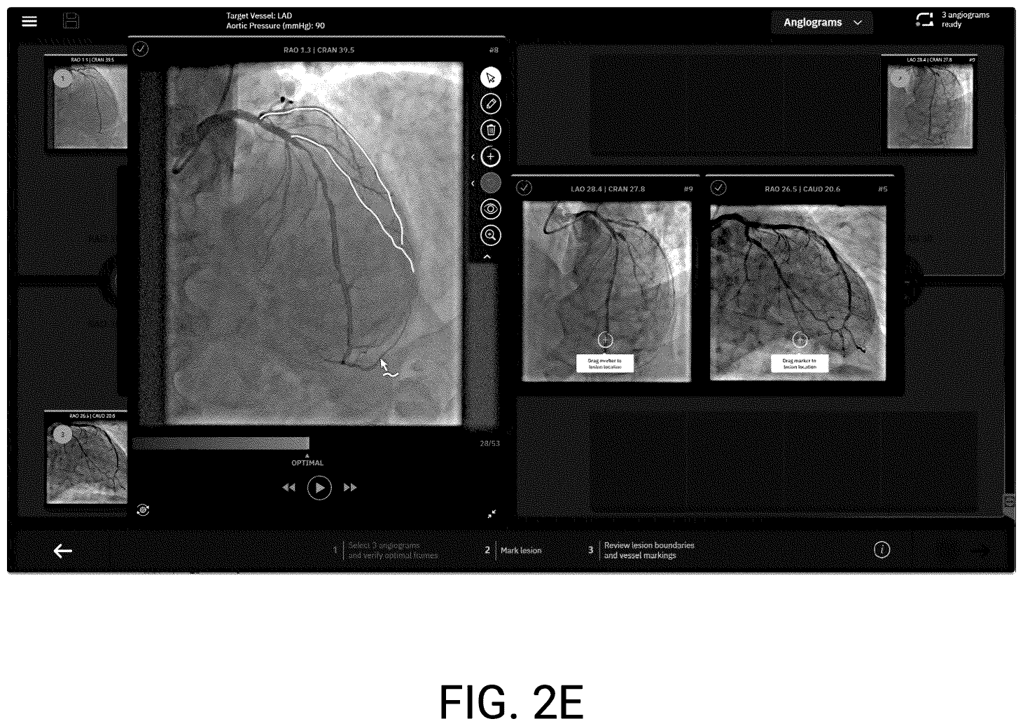

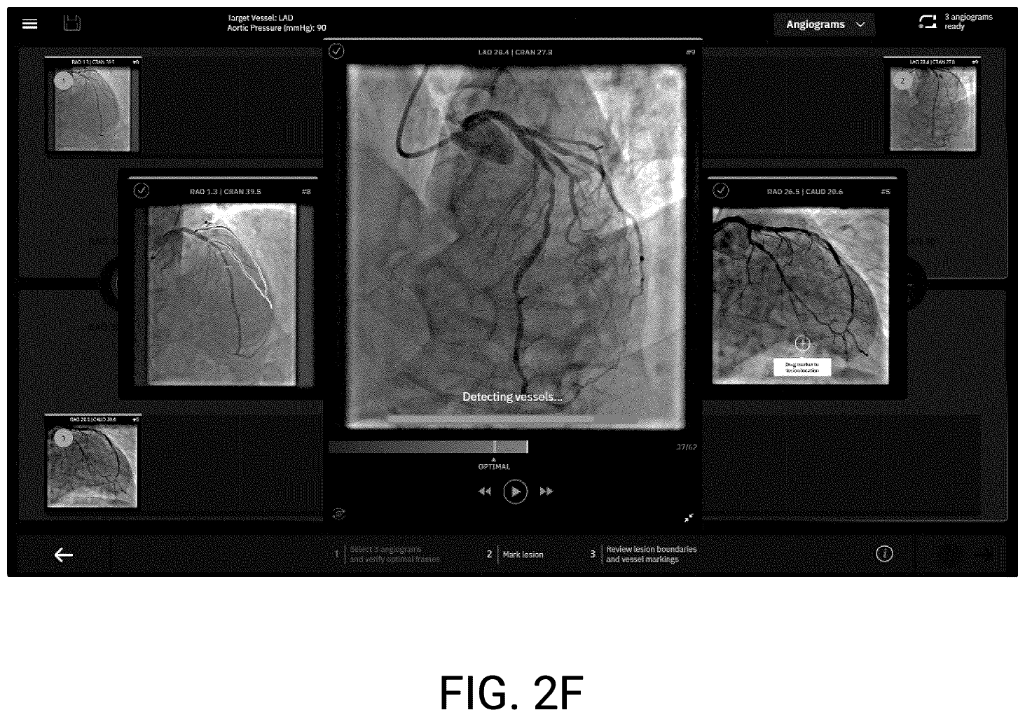

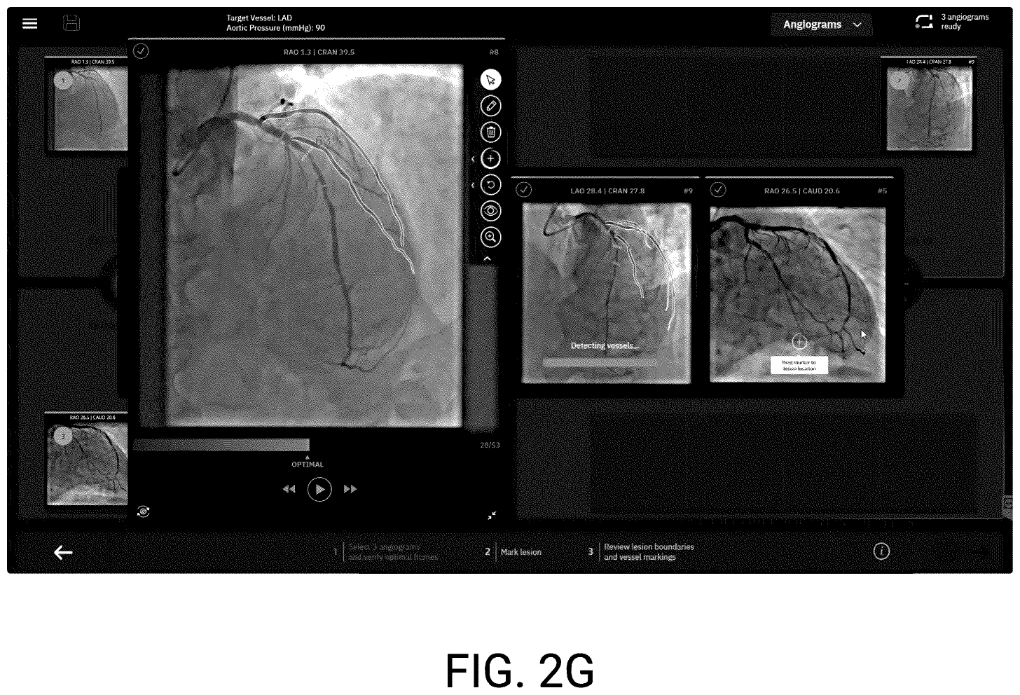

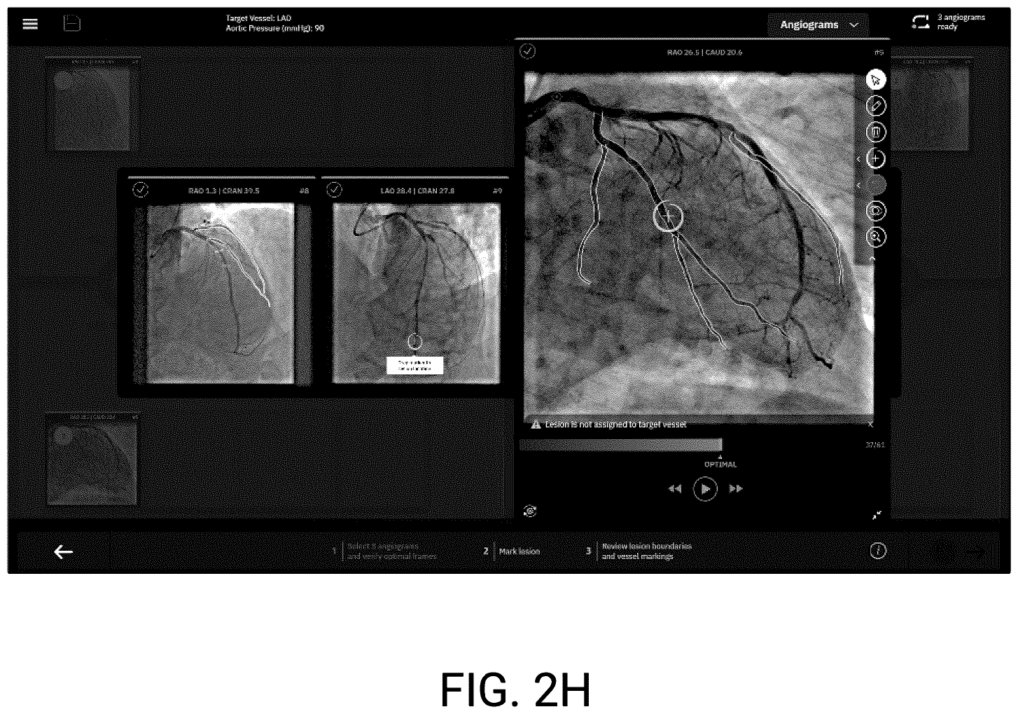

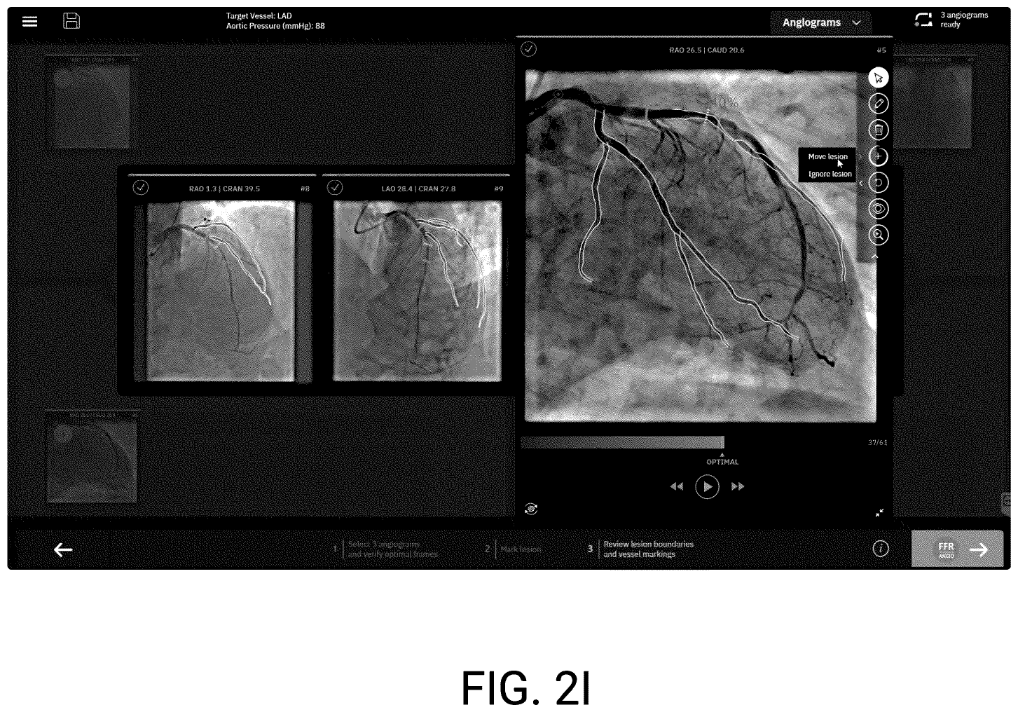

As will be described, the medical professional may identify a lesion, or set of lesions, which is depicted in each accessed medical image. For example, and as illustrated in FIG. 2C, user input may be provided to a medical image which indicates a portion of the patient's heart that has a lesion. Based on this user input, the system may then identify vessels of the patient's heart which are associated with the lesion. As an example, the vessels may represent vessels which are associated with a same path through the patient's heart as an identified lesion (e.g., the lesion may be included in a vessel or vessels associated with the path). For example, FIG. 2D illustrates example vessels associated with the lesion identified in FIG. 2C. In this example, the figure illustrates vessel markings (e.g., lumen contours) which correspond to vessels detected based on medical image.

The medical professional may prefer to adjust the vessels identified by the system. For example, and as illustrated in FIG. 2E, the medical professional may provide user input to extend vessel markings (e.g., lumen contours) that correspond with the identified vessels. In the illustrated example, the medical professional has extended an identified vessel via dragging a graphical element (e.g., vessel markings) representing the identified vessel. Thus, adjusting the vessels may include adjusting the length, or other geometric characteristics, of vessel markings (e.g., increasing or decreasing a size or other geometric characteristic associated with a vessel). Adjusting the vessel markings may also include, for example as described, adding an additional vessel or vessel segment as being relevant to a lesion. Adjusting the vessel markings may also include, for example, removing a vessel or vessel segment as not being relevant. For example, the system may have misclassified a vessel as having blood flow which goes into, or extends from, a lesion.

In this way, the medical professional may prepare information for the system to use, such as via adjusting the medical images, to generate a three-dimensional model and/or index indicative of vascular function. For example, in some embodiments the system may generate a three-dimensional model based, at least in part, on matching features depicted in the medical images. The system may determine geometry information for the patient's heart, such as assigning diameters and/or radii to the identified vessels. Based on the geometry information, the system may determine an FFR value, or FFR values, associated with a lesion. For example, the FFR values may be determined across the length of the vessels such that a drop in FFR across the lesion may be determined.

Advantageously, the techniques described herein allow the medical professional to adjust the medical images in a substantially parallel manner. As an example, the medical professional may individually identify a lesion on each medical image presented in a user interface (e.g., 2, 3, 4, and so on medical images presented in the same user interface, for example at the same time). For this example, while the system is determining vessels based on an identified lesion in a first image (e.g., the identification of the lesion may trigger vessel marking determinations in the first image), the medical professional may interact with the remaining images. As an example, the medical professional may identify a lesion in a second image, adjust the vessels in a third image for which the vessels were already determined by the system, and so on.

Thus, and as will be described, a medical professional may use a same user interface to perform, in parallel, at least any of the following operations:
Selection of a medical image from a particular viewpoint,
Identification of a lesion on a medical image,
Initiation of system detection of vessels and vessel boundaries,
Adjustment of vessels detected on a medical image which has had a lesion identified,
Adjustment of a lesion after it has been identified.

For example, the medical professional may individually interact with, and perform any of the above-identified operations on, each medical image using the same user interface. As one example, a medical professional may identify a lesion on a first medical image. As the system is detecting vessels for the first medical image, the medical professional may identify a lesion on a second medical image. The medical professional may adjust the detected vessels on the first image as the vessels are being detected on the second medical image. The medical professional may identify a lesion on a third medical image, and as the vessels are being detected on the third medical image the medical professional may identify a new lesion on the first medical image.

Prior techniques required the medical professional to interact with multiple user interfaces in discrete steps. For example, the medical professional was required to select angiographic images using a first user interface. In this example, the medical professional would then identify lesions on the angiographic images using the first user interface. Subsequently, the medical professional would select a user interface element (e.g., a button) to trigger detection of vessels. The detected vessels would then be presented using a distinct, second, user interface. The medical professional could then adjust the vessels using the interface. Thus, the medical professional would have to identify and mark the lesion of interest for all medical images before prompting the system to begin detection of vessels and before he/she could adjust the vessels. Additionally, to adjust the position of the lesion the medical professional would be required to back out of the second user interface and return to the first user interface. The medical professional would then select a new lesion and have to re-trigger the detection of vessels to move back to the second user interface.

The above-described parallel operations will be described in more detail below with respect to FIGS. 2A-2I.

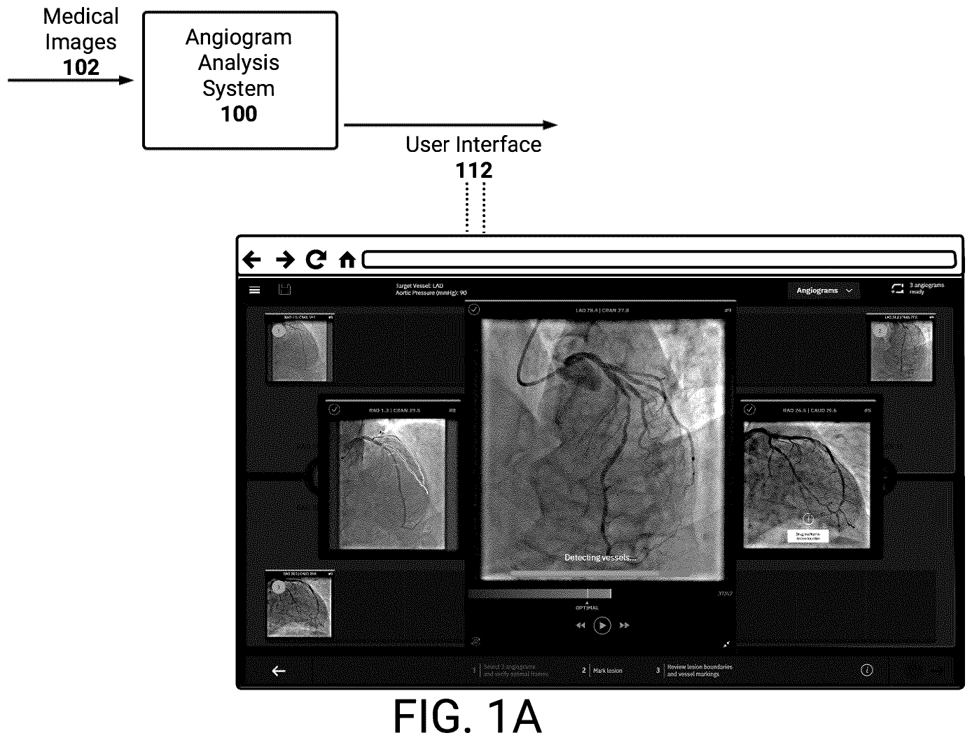

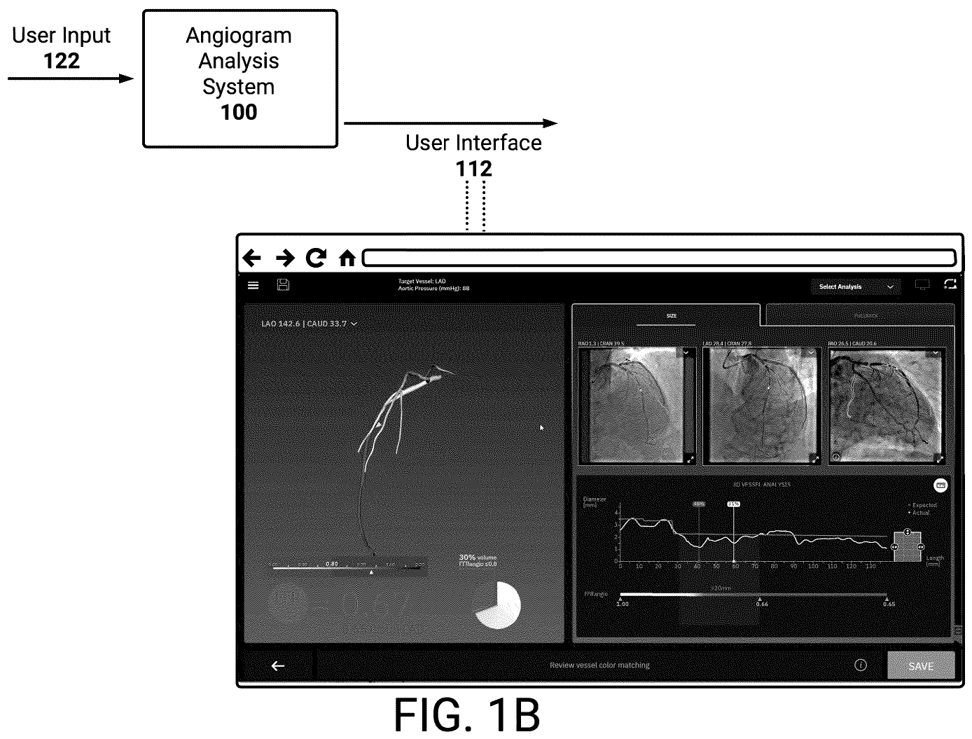

FIG. 1A is a block diagram of an example angiogram analysis system 100 generating a user interface 112. The angiogram analysis system 100 may represent a system of one or more processors, such as a user device, a computer, a back-end server system, and so on. As described above, the system 100 may receive medical images 102 (e.g., angiograms) for analysis. The system 100 may additionally present a user interface 112 for interaction with by an end-user (e.g., a medical professional). The system 100 may, in some embodiments, present the user interface 112 via a display of the system. The system 100 may also cause presentation of the user interface 112 via a display of a different system or user device (e.g., the user interface 112 may represent a front-end of a web application).

The user interface 112 illustrated in FIG. 1A may be used by the end-user to prepare medical images 102 for further analysis by the system 100. For example, the end-user may identify locations of lesions in the user interface 112. The end-user may additionally cause detection of vessels based on the lesions (e.g., detection of vessel markings for inclusion in the user interface 112, such as lumen contours presented, or otherwise layered, over an angiogram). The end-user may additionally adjust the detected vessels, for example to add, remove, or adjust the length or other geometrical characteristics of the vessels. Advantageously, the end-user may use the same user interface 112 to perform these actions in parallel. Example parallel actions are described below with respect to FIGS. 2A-2I.

FIG. 1B is a block diagram of the example angiogram analysis system 100 updating the user interface 112 based on user input 122. As described above, the end-user may provide user input 122 to select lesion(s), cause detection of vessels, adjust vessels, update location of a previously-selected lesion, and so on. Subsequently the angiogram analysis system 100 may determine an index indicative of vascular function (e.g., FFR) and/or a three-dimensional model of a portion of a heart (e.g., a portion associated with the selected lesion(s)).

With respect to the three-dimensional model, the model may indicate diameters associated with vessels included in the patient's heart. In this way, the system 100 may compute the FFR value based on the model. Additional description related to the above is included in U.S. Pat. No. 10,595,807, which is hereby incorporated herein by reference in its entirety.

FIGS. 2A-2I illustrate example user interfaces associated with parallel adjusting of angiograms. These user interfaces may represent user interfaces presented via the system described herein, and the user interfaces may not be necessarily linked to each other in the order shown in FIGS. 2A-2I.

FIG. 2A illustrates an example user interface usable to select a target vessel of a heart. In the illustrated example, a medical professional may select from particular vessels. For example, the medical professional has selected the left anterior descending (LAD) vessel for analysis. Subsequently, the user interface may update to enable selection of particular medical images which depict the vessel.

FIG. 2B illustrates a user interface which enables selection of medical images, such as angiographic images. In the illustrated example, the user interface indicates that the medical professional is to select three angiograms. As described above, and as incorporated herein, the medical images may depict the selected vessel from different viewpoints. Information from these viewpoints may be combined to form a three-dimensional model. In the upper left, right, and lower left, example medical images are shown. Each medical image may represent an image from a multitude of images which were taken at a particular viewpoint.

FIG. 2C illustrates the user interface presenting three medical images which have been selected for analysis. The medical professional, in the illustrated example, has selected the left-most image to provide additional user input to. In response, the left-most image has enlarged to, for example, show detail and provide a visual cue that it can be interacted with. The left-most image may represent the ‘optimal’ image which is selected from a multitude of medical images at the same, or substantially similar, viewpoint. Determining the optimal image is described in more detail with respect to U.S. Patent Pub. 2023/0252632, which is hereby incorporated herein by reference in its entirety. The medical professional may additionally interact with the left and/or right arrow below the left-most image to select a different medical image at the viewpoint.

The user interface enables the medical professional to select a lesion, or lesions, on the left-most image. In the example, the medical professional has selected a lesion (e.g., the lesion marker, such as the plus sign inside of the circle). The system described herein has then initiated the process to detect vessels associated with the lesion.

Advantageously, while the system is detecting vessels, the medical professional may perform one or more of the following:
Choose another lesion location. This may be advantageous, as an example, since the automation to detect vessels may trigger once the lesion location identified (e.g., even if at a wrong location).
View or change to an alternate medical image while the vessel detection is in process. For example, the medical professional may select the middle image which may enlarge to present detail while the left-most image returns back to its prior size (e.g., standard size).
Select other medical images for analysis.
Identify lesions on other medical images.
Edit vessels on a different medical image (e.g., the right medical image may have already had its vessels detected based on a prior identification of a lesion).

FIG. 2D illustrates the left-most medical image with its vessels having been detected. For example, the vessels are illustrated via vessel markings. In some embodiments, the vessel markings may include lumen contours which may be reflective of an internal shape associated with a vessel or vessels.

The medical professional may thus edit these vessels, for example as illustrated in FIG. 2E. For example, the medical professional may adjust the vessel by dragging a graphical marker or indicia (e.g., vessel markings, such as an outline as illustrated) along a portion of the image. As another example, the medical professional may provide user input (e.g., touches, mouse clicks, and so on) on portions of the image to cause vessel markings to extend between the portions. Thus, the medical professional may adjust vessel markings to update geometric characteristic(s) associated with a vessel. As an example, the medical professional may adjust a lumen contour or lumen contours.

Advantageously, the user interface may enable the medical professional to adjust the vessel markings with less than all of the medical images having received user input to identify lesions. For example, the left-image may have been interacted with first by the medical professional. Thus, the remaining medical images, or at least one medical image, may not have received input to identify lesions. However, the medical professional may still edit the left-most medical image. Furthermore, the user interface may enable the medical professional to adjust the vessel markings while vessels are being detected in a different medical image (e.g., the medical professional may have identified a lesion in a different image and then started adjusting vessel markings in the left-most image). The user interface may enable the medical professional to adjust the location of the lesion in the different image while the processing to identify vessel markings has not yet been completed for the identified lesion.

Additionally, the medical professional may perform actions on the remaining two selected medical images. For example, the medical professional may select, or otherwise identify a location of, a lesion, update a previously selected lesion, edit vessels, and so on, in parallel (e.g., without pausing processing or actions being performed on remaining images). In this way, using the same user interface the medical professional may perform actions on any of the medical images. The medical professional may additionally finalize one or all of the medical images.

FIG. 2F illustrates an example of the medical professional performing parallel actions associated with preparing information for analysis by the system. In the illustrated example, the left-most image illustrates that the automatic vessel detection process has been completed. The middle image illustrates that the vessels are presently being detected. FIG. 2G illustrates FIG. 2F after vessels have been detected.

The user interface of FIG. 2F illustrates that the medical professional is providing user input to the middle image to view additional medical images from the same viewpoint. For example, FIG. 2F illustrates the medical professional has moved beyond the ‘optimal’ image and is instead viewing a medical image later in the set of images at the same viewpoint.

The user interface illustrates the right-image as being selected but a lesion location has not yet been specified.

FIG. 2H illustrates that the right-image has an incorrect lesion which is not on the target vessel. For example, the lesion may not correspond to the currently selected target vessel (e.g., LAD vessel as described above). This may alert the medical professional of the incorrect location, such as prior to doing vessel detection for all medical images thus allowing the user to modify the lesion location (e.g., adjust a lesion marker).

Advantageously, the user interface may enable the medical professional to adjust the lesion with less than all of the medical images having received user input to identify lesions. For example, while not illustrated the right-image may have been interacted with first by the medical professional. Thus, the remaining medical images, or at least one medical image, may not have received input to identify lesions.

In contrast, prior techniques required the medical professional to select the lesion location for the three medical images via a first user interface. If the error was presented in a second user interface, for example after the medical professional selected a button to proceed, the medical professional was required to go back to a step (e.g., to the first user interface) to modify the lesion location.

FIG. 2I illustrates the medical professional moving a lesion. For example, the medical professional may adjust a lesion as described above (e.g., with respect to FIG. 2E).

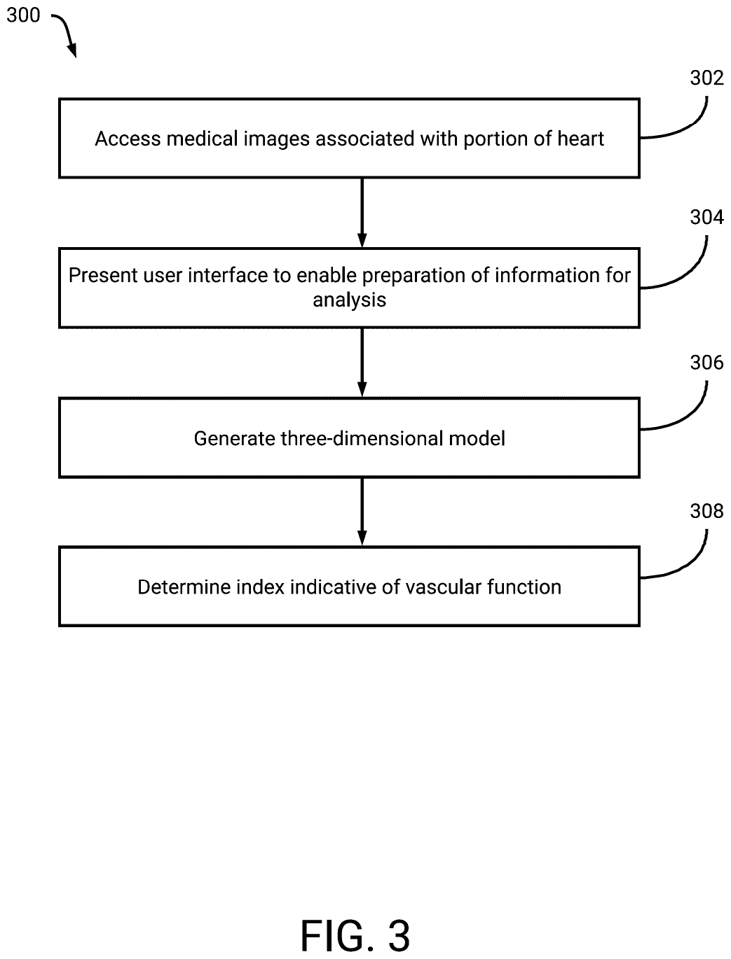

FIG. 3 is a flowchart of an example process 300 for parallel adjusting of angiograms prior to generation of an index indicative of vascular function and/or three-dimensional cardiac model. For convenience, the process 300 will be described as being performed by a system of one or more computers (e.g., the angiogram analysis system 100).

At block 302, the system accesses medical images depicting a portion of a patient's heart. As described above, medical images (e.g., angiographic images) may be obtained of the portion of the patient's heart. For example, an imaging system (e.g., a c-arm) may be used which rotates about the patient.

At block 304, the system presents a user interface which allows for preparation of information for analysis. The user interface may represent a unified user interface which allows the user to select medical images for analysis. For example, the system may identify optimal image frames from a set of image frames at specific viewpoints of the imaging system. The user may then perform, in parallel, actions via the user interface. As described above, the user may individually interact with each selected image and (1) select a lesion of interest, (2) initiate the detection of vessels, (3) update the detected vessels, (4) select a different image frame, (5) select a new lesion location, and so on.

The user may select a lesion on a first medical image and then adjust the detected vessels (e.g., adjust corresponding vessel markings). The user may adjust the detected vessels without navigating to a later user interface. The user may adjust the detected vessels without requiring selection of a lesion on other medical images. The user may adjust the detected vessels while the system is detecting vessels on a different medical image. The user may select a new lesion without requiring selection of lesions on other medical images. The user may select the new lesion while the system is detecting vessels on a different medical image. The user may prepare the information required to determine a three-dimensional model and/or index indicative of vascular function using the same user interface.

At block 306 and block 308 the system generates a three-dimensional model and an index (e.g., an FFR value). The information provided via the user interface, such as the selected medical images and user input to identify lesions, adjust vessels, and so on, may be used to determine the model and index.

### Other Embodiments

All of the processes described herein may be embodied in, and fully automated, via software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence or can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and engines described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, the term “about” refers to within ±10%.

The terms “comprises”, “comprising”, “includes”, “including”, “having”, “such as” and their conjugates mean: “including but not limited to”.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be emphasized that many variations and modifications may be made to the above-described embodiments, the elements of which are to be understood as being among other acceptable examples. All such modifications and variations are intended to be included herein within the scope of this disclosure.

What is claimed is:

1. A method implemented by a system of one or more computers, the method comprising:
providing, for presentation via an interactive user interface, a plurality of angiographic images, wherein the interactive user interface enables receipt of user input that causes parallel processing of: (1) identification of a lesion on an individual angiographic image, wherein identification of the lesion triggers detection of vessel markings associated with vessels depicted in the individual angiographic image, and (2) adjustment of vessel markings associated with one or more vessels detected in a different individual angiographic image in which a lesion was identified;
receiving user input to adjust vessel markings associated with one or more vessels detected in a first angiographic image of the plurality of angiographic images, wherein the interactive user interface is configured to display the adjustment with less than all of the plurality of angiographic images having received user input to identify respective lesions; and
updating the interactive user interface based on the user input, wherein the interactive user interface is configured to trigger determination of an index indicative of vascular function based on user input indicating completion.

2. The method of claim 1, wherein adjustment of vessel markings comprises adjustment of lumen contour.

3. The method of claim 2, wherein the lumen contour is reflective of an internal shape associated with the one or more vessels.

4. The method of claim 1, wherein a vessel marking is a graphical element representing a vessel.

5. The method of claim 1, wherein adjustment of vessel markings comprises adjusting a geometric characteristic associated with a vessel.

6. The method of claim 1, further comprising:
receiving subsequent user input to adjust a location of a lesion identified on the first angiographic image,
wherein less than all of the plurality of angiographic images received user input to identify respective lesions; and
triggering updated detection of vessel markings included in the first angiographic image based on the adjusted location of the lesion.

7. The method of claim 1, further comprising detecting vessel markings in the first angiographic image based on identification of a lesion, wherein the vessel markings are detected based on blood flow which goes into, or extends from, the lesion.

8. The method of claim 1, wherein the user input to adjust the vessel markings detected in the first angiographic image is received while vessel markings are being detected in a second angiographic image in response to a lesion being identified on the second angiographic image.

9. The method of claim 8, wherein while vessel markings are being detected in the second angiographic image, user input is received to adjust the lesion, and wherein the user input triggers updated detection of vessel markings based on the adjusted lesion.

10. The method of claim 1, wherein determination of the index indicative of vascular function is, at least in part, in response to detection of vessel markings in all of the plurality of angiographic images.

11. The method of claim 1, wherein each angiographic image included in the plurality of angiographic images is associated with a respective imaging angle, wherein each angiographic image is automatically selected from a set of angiographic images associated with a same imaging angle, and wherein interactive user interface further enables parallel processing of changing an individual angiographic image associated with a particular imaging angle to a different angiographic image associated with the particular imaging angle.

12. The method of claim 1, wherein the user input indicating completion reflects user input to transition to the updated interactive user interface in which the index indicative of vascular function is included.

13. A system comprising one or more processors and non-transitory computer storage media storing instructions that when executed by the one or more processors, cause the one or more processors to perform operations comprising:
providing, for presentation via an interactive user interface, a plurality of angiographic images, wherein the interactive user interface enables receipt of user input that causes parallel processing of: (1) identification of a lesion on an individual angiographic image, wherein identification of the lesion triggers detection of vessel markings associated with vessels depicted in the individual angiographic image, and (2) adjustment of vessel markings associated with one or more vessels detected in a different individual angiographic image in which a lesion was identified;
receiving user input to adjust vessel markings associated with one or more vessels detected in a first angiographic image of the plurality of angiographic images, wherein the interactive user interface is configured to display the adjustment with less than all of the plurality of angiographic images having received user input to identify respective lesions; and
updating the interactive user interface based on the user input, wherein the interactive user interface is configured to trigger determination of an index indicative of vascular function based on user input indicating completion.

14. Non-transitory computer storage media storing instructions that when executed by a system of one or more processors, cause the one or more processors to perform operations comprising:
providing, for presentation via an interactive user interface, a plurality of angiographic images, wherein the interactive user interface enables receipt of user input that causes parallel processing of: (1) identification of a lesion on an individual angiographic image, wherein identification of the lesion triggers detection of vessel markings associated with vessels depicted in the individual angiographic image, and (2) adjustment of vessel markings associated with one or more vessels detected in a different individual angiographic image in which a lesion was identified;
receiving user input to adjust vessel markings associated with one or more vessels detected in a first angiographic image of the plurality of angiographic images, wherein the interactive user interface is configured to display the adjustment with less than all of the plurality of angiographic images having received user input to identify respective lesions; and
updating the interactive user interface based on the user input, wherein the interactive user interface is configured to trigger determination of an index indicative of vascular function based on user input indicating completion.

15. A method performed by a system of one or more computers for parallel adjusting of medical images, the method comprising:
accessing medical images depicting a portion of a patient's heart; and
presenting a unified user interface which allows for a user to select medical images for analysis, wherein the unified user interface allows a user to select a lesion and adjust detected vessels on a first medical image while the system is detecting vessels on a different medical image displayed on the unified user interface without navigating to a later user interface.

16. The method of claim 15, wherein the unified user interface allows the user to adjust the detected vessels on the first medical image without requiring selection of a lesion on other medical images.

17. The method of claim 15, wherein the unified user interface allows the user to select a new lesion on the first medical image without requiring selection of lesions on other medical images.

18. The method of claim 15, wherein the unified user interface allows the user to select a new lesion on the first medical image while the system is detecting vessels on the different medical image.

19. The method of claim 15, further comprising generating a three-dimensional model using the selected medical images.

20. The method of claim 15, further comprising determining an index indicative of vascular function using the selected medical images.

21. A system comprising one or more processors and non-transitory computer storage media storing instructions that when executed by the one or more processors, cause the one or more processors to perform the method of claim 15.

22. Non-transitory computer storage media storing instructions that when executed by a system of one or more processors, cause the one or more processors to perform the method of claim 15.
